SEQUENCE LISTING (6 sequences)

SEQ ID NO: 1 - protein, 235 aa, Artificial Sequence
  Artificially Synthesized Sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 274 aa, Artificial Sequence [sequence omitted]

SEQ ID NO: 3 - protein, 312 aa, Artificial Sequence
  Artificially Synthesized Sequence
RMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - DNA, 745 bp, Artificial Sequence
  Artificially Synthesized Sequence
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 5 - DNA, 822 bp, Artificial Sequence
  Artificially Synthesized Sequence
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctctgtacggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagc

SEQ ID NO: 6 - DNA, 939 bp, Artificial Sequence
  Artificially Synthesized Sequence
aggatgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctaa

(19) United States
(12) Patent Application Publication
(10) Pub. No.: US 2021/0317184 A1
(43) Pub. Date: Oct. 14, 2021

(54) T CELL MODIFICATION

(71) Applicants: GlaxoSmithKline Intellectual Property Devolopment Limited, Brentford Middlesex (GB); ADAPTIMMUNE LTD, Abingdon, Oxfordshire (GB)

(72) Inventors: Sara Jane BRETT, Stevenage (GB); Marie L. DAVIES, Stevenage (GB); Thomas SOUTHGTE, Stevenage (GB); Katherine ADAMS, Abingdon, Oxfordshire (GB); Luca MELCHIORI, Abingdon, Oxfordshire (GB); Louise RICE, Abingdon, Oxfordshire (GB)

(21) Appl. No.: 17/272,865

(22) PCT Filed: Sep. 5, 2019
(86) PCT No.: PCT/IB2019/057485

§ 371 Date: Mar. 2, 2021

Related U.S. Application Data
(60) Provisional application No. 62/727,103, filed on Sep. 5, 2018.

(51) Int. Cl. C07K 14/705; C07K 14/725; A61K 35/17; C12N 15/86

(52) U.S. Cl. CPC C07K 14/70517; C07K 14/7051; C12N 2740/15043; C12N 15/86; A61K 35/17

(57) ABSTRACT

Improved compositions and methods for treating diseases, such as cancer, by providing a cell immunotherapy, wherein the cell immunotherapy is an immunomodulatory cell expressing an exogenous CD8 co-receptor and a modified T cell receptor (TCR) are provided. Also provided are polynucleotides, expression vectors, and immunomodulatory cells including the immunotherapy, as well as methods of generating said immunomodulatory cells.

### FIELD OF THE INVENTION

The present invention relates generally to modification of T cells to increase their cytotoxic activity and the use of modified T cells in immunotherapy, for example, for the treatment of cancer.

### BACKGROUND TO THE INVENTION

Immunotherapeutics are poised to transform the cancer treatment landscape with the promise of long-term survival (McDermott et al., Cancer Treat Rev. 2014 October; 40(9): 1056-64). There is a clear unmet medical need for new immunomodulatory drugs to expand patient population and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015 Apr. 9; 161(2): 205-14). This typically requires tumor specific CD4+ and CD8+ T-cells recognising tumor-associated peptide antigens presented by MHC molecules. Different vaccination strategies and adoptive transfer of ex vivo expanded tumor infiltrated lymphocytes have in some cases demonstrated the ability of tumor specific T-cells to treat late stage cancer (Rosenberg et al., Nat Med. 2004 September; 10(9): 909-15). However high tolerance to tumor antigens combined with the potent immunosuppressive microenvironment often present at the tumor site manifests in suboptimal activation of T cell anti-tumor activity. Thus, individuals lacking high affinity T-cells may not respond to immune checkpoint blockade therapies, such as anti-PD-1 and anti-CTLA-4, due to T-cell tolerance to self-antigens.

Genetic engineering may help to overcome the problems of the low frequency of endogenous high affinity T cells to tumor antigens by the generation of high affinity T cell receptors (TCR), and provide clinical benefit to patients who do not respond to treatment with checkpoint inhibitors. This approach has been shown to increase the affinity of the wild type TCRs for their natural ligand peptide/MHC class I complex 10-1000 fold in vitro for several antigens including gp100, MAGE-A3 and NY-ESO-1 (Li et al., Nat Biotechnol. 2005 March; 23(3): 349-54; Robbins et al., J Immunol. 2008 May 1; 180(9): 6116-31.). Higher affinity TCRs allow T cells to respond to lower levels of antigen; this is important where tumor microenvironment has adapted to reduce antigen expression and decrease expression of MHC class I molecules (Barrett and Blazar, N Engl J Med. 2009 Jul. 30; 361(5): 524-5; Marincola et al., Adv Immunol. 2000; 74: 181-273). Redirecting T cells towards tumors has been achieved via TCR-engineered T cell therapies or with T-cell-redirecting biologics (Bossi et al., Cancer Immunol Immunother. 2014 May; 63(5): 437-48; Fan et al., J Hematol Oncol. 2015 Dec. 21; 8: 130).

T cell therapy has shown curative potential for treatment of some recurrent or high risk tumors (Dudley et al., J Immunother. 2003 July-August; 26(4): 332-42; Dudley et al., J Clin Oncol. 2005 Apr. 1; 23(10): 2346-57; Kalos et al., Sci Transl Med. 2011 Aug. 10; 3(95): 95ra73). There are currently two methods being used to genetically engineer patient T cells to recognise tumor antigens including chimeric antigen receptors (CARs) and affinity matured TCRs.

However, CARs are restricted to targeting only epitopes on the cell surface. TCR-based therapeutics can recognise not only cell surface proteins, but also internal cell proteins. In addition, the TCR approach more closely mimics the natural function of the T cell by recruiting the endogenous signalling molecules and spatial-temporal interactions between T cells and their specific targets. It is, however, restricted to individuals who share the appropriate MHC restriction, recognised by the TCR. This type of therapy will require the parallel development of patient selection assays for both the HLA type and the antigen expression.

The binding of a MHC Class I-restricted T cell receptor (TCR) to the peptide-MHC complex is stabilized by a glycoprotein called CD8 (cluster of differentiation 8), which also recruits the Src-family kinase Lck, and potentiates signalling. CD8 binding to the constant portion of MHC class I results in increased affinity of binding and decreased threshold of response to antigen on target cells (Gao, Nature. 1997 Jun. 5; 387(6633): 630-4; Artyomov et al., Proc Natl Acad Sci USA. 2010 Sep. 28; 107(39): 16916-21). Addition of a CD8 transgene into a TCR lentiviral vector could confer to CD4+ T cells a similar increased response, augmenting their ability to provide helper function to CD8+ T cells as well as additional direct tumor cell killing, possibly resulting in enhanced clinical efficacy.

CD8α/CD8β (cluster of differentiation 8) is a heterodimeric transmembrane glycoprotein expressed by cytotoxic T cells, natural killer (NK) cells and dendritic cells. It binds to conserved regions on Class I peptide-Major Histocompatibility antigens (pMHCs, in man these are normally described as peptide-Human Leucocyte Antigens or pHLAs) and in doing so it acts as a generic co-receptor for MHC peptide-specific binding by T Cell Receptors (TCRs). CD8α/CD8 is not found in mature CD4+ T cells where their antigen-specific TCRs bind to the related but different Class II pMHC antigens and where the CD4 homodimer acts as the TCR co-receptor.

The most common type of co-receptor-dependent TCRs are heterodimeric transmembrane glycoproteins with an α and β polypeptide chain. When α/β TCRs bind Class I pMHC antigens they trigger an intracellular signalling cascade of phosphorylation events that activate a plethora of cellular events including the killing of pMHC-expressing target cells by cytotoxic T cells. This signalling cascade is initiated by the phosphorylation of TCR-bound CD3 transmembrane proteins by Lck (Lymphocyte-specific protein tyrosine kinase). Intracellular associations between CD8α/CD8β and Lck are thought to potentiate TCR signalling.

In humans, in addition to the CD8α/CD8β heterodimer, approximately one third of CD8+ cells also display a CD8α/CD8α homodimeric form. In some intestinal T cells, NK cells, and γ/δ T cells, only this homodimeric form is found. Evidence suggests that in humans, this CD8α homodimer could fully functionally substitute for the CD8α/CD8β heterodimer (Cole et al., Immunology. 2012 October; 137(2): 139-48).

In vivo, the concurrent binding of TCRs and CD8 dimers to Class I pHLA impacts on the thymic positive/negative selection of T cell clones. This dictates the pHLA antigen affinity of the TCRs expressed by these T cell clones. In general, the TCR antigen affinities in pathogen-associated pHLA-reactive T cell clones are higher than the equivalent T cell clones that recognize cancer-associated antigens. TCR affinity enhancement technologies can increase the affinity of cancer-reactive TCRs to close to that of pathogen-reactive TCRs. These increases in TCR affinity result in TCRs that are usually CD8 co-receptor independent. Cellular transduction of CD4+ T cells with gene expression vectors that express these TCRs creates a novel entity of Class I pHLA-specific CD4+ T cells with killer and helper functions which otherwise could only normally be activated by Class II-specific peptide-antigens (Tan et al., Clin Exp Immunol. 2017 January; 187(1): 124-137). These TCRs allow T cells to more efficiently recognize their cancer target cells than do their wildtype parent TCRs. Importantly, pHLA antigen specificity is maintained even in CD8+ T cells, i.e., in the presence of endogenous CD8 co-receptors.

Although co-receptor independence means that these affinity-enhanced TCRs can also function to an extent in CD4+ T cells it is clear that the optimum TCR affinity in CD4+ T cells is higher than it is in CD8+ T cells (Tan et al.).

There is an ongoing need for new and improved TCR-based therapeutics to enhance the magnitude and duration of anti-tumor responses in patients.

### SUMMARY OF THE INVENTION

The present invention provides, in a first aspect, modified T cells that present an exogenous CD8 co-receptor or fragment thereof, and a T cell receptor (TCR). In one embodiment of the first aspect, the modified T cells may comprise a nucleic acid construct that comprises (i) a first nucleotide sequence encoding a CD8 co-receptor or fragment thereof, and (ii) a second nucleotide sequence encoding a T cell receptor (TCR).

The present invention also provides, in a second aspect, a pharmaceutical composition comprising a plurality of modified T cells of the first aspect of the invention that present a CD8 co-receptor or fragment thereof, and a TCR, and a pharmaceutically acceptable carrier.

The present invention also provides, in a third aspect, methods of treating cancer in a human comprising administering a pharmaceutical composition of the second aspect of the invention to said human.

The following detailed description of preferred embodiments of the invention will be better understood when read in conjunction with the appended drawings. For the purpose of illustrating the invention, there are shown in the drawings, embodiments which are presently preferred. It should be understood, however, that the invention is not limited to the precise arrangements and instrumentalities of the embodiments shown in the drawings.

### DESCRIPTION OF DRAWINGS/FIGURES

FIG. 1 shows a schematic of overlapping PCR strategy used to generate full length CD8α_F2A_NY-ESO^{c259} TCR coding sequence.

FIG. 2 shows the plasmid map for a CD8α NY-ESO-1^{c259} TCR transfer plasmid.

FIG. 3 shows activation of T cells in response to antigen, as measured by CD40L expression in nontransduced (ntd) T cells, CD8α NY-ESO^{c259} T, or NY-ESO^{c259} CD8α NY-ESO^{c259} T cells.

FIG. 4 shows the proliferation of CD4+Vbeta+ and CD4+Vbeta− T cell subsets within ntd, NY-ESO^{c259} T, or CD8α NY-ESO^{c259} T cells in response to antigen positive (A375) and negative (HCT-116) cell lines.

FIG. 5 shows proliferation index data from three donor wavebags of CD8+Vbeta+ and CD4+Vbeta+ T cell subsets within ntd, NY-ESO-1^{c259} T, or CD8α NY-ESO-1^{c259} T cells in response to antigen positive cell line A375

FIG. 6 shows IL-2 release analysis by Luminex™ MAGPIX® assay for ntd, NY-ESO-1^{c259} T, or CD8α NY-ESO-1^{c259} T cells upon stimulation with NY-ESO-1 peptide (SLLMWITQC).

FIG. 7 shows IFN-γ release by ntd, NY-ESO-1^{c259} T, or CD8α NY-ESO-1^{c259} T cells in co-culture with NY-ESO-1-positive and negative A375 GFP 3D spheroids.

FIG. 8 shows granzyme B release when NY-ESO-1 positive cells were cultured with ntd, NY-ESO-1^{c259} T, or CD8α NY-ESO-1^{c259} T cells.

FIG. 9 shows granzyme B release assay data in 3D cell culture assay (ntd, NY-ESO-1^{c259} T, or CD8α NY-ESO-1^{c259} T cells in 3D culture of A375-GFP cells)

FIG. 10 shows cell-killing over time of A375 melanoma cells by NY-ESO-1^{c259} T, or CD8a NY-ESO-1^{c259} T cells from a single donor.

FIG. 11 shows area under the curve (AUC) analysis of the cytotoxicity activity of CD8a NY-ESO-1^{c259} T cells compared with NY-ESO-1^{c259} T cells against A375 target cells when co-incubated with A375 target cells over a time frame of 0-51 hours for 7 donors.

FIG. 12 shows IncuCyte killing experiments on Mel624 cells of CD8α NY-ESO-1^{c259} T cells compared with NY-ESO-1^{c259} T cells.

FIG. 13 shows cytotoxic activity of Wave147 and Wave149 CD8α NY-ESO-1^{c259} T cells towards NY-ESO-1 expressing A375-GFP 3D spheroids (large ˜500 μm diameter).

### DETAILED DESCRIPTION OF THE INVENTION

The present invention provides modified T cells that present an exogenous CD8 co-receptor or fragment thereof, and a T cell receptor (TCR). In one embodiment, the CD8 co-receptor is a CD8α homodimer. In another embodiment, the CD8 co-receptor comprises an amino acid sequence having at least 80%, 85%, 90%, 95%, 96%, 97%, 98%, 99%, or 100% sequence identity to CD8α (SEQ ID NO: 1).

The amino acid sequence of CD8α is shown in SEQ ID NO:1.

(SEQ ID NO: 1)
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV.

In another embodiment, the TCR is affinity matured. In another embodiment, the TCR comprises an α chain and a β chain. In another embodiment, the TCR is a NY-ESO-1 TCR. NY-ESO-1^{c259} is an affinity enhanced TCR, mutated at positions 95 and 96 of the alpha chain 95:96LY relative to the wildtype TCR. NY-ESO-1^{c259} binds to a peptide corresponding to amino acid residues 157-165 of the human cancer testis Ag NY-ESO-1 (SLLMWITQC) in the context of the HLA-A2+ class 1 allele with increased affinity relative to the unmodified wild type TCR (Robbins et al J Immunol (2008) 180(9):6116).

In another embodiment, the amino acid sequence of the NY-ESO-1 TCR comprises an amino acid sequence having at least 80%, 85%, 90%, 95%, 96%, 97%, 98%, 99%, or 100% sequence identity to NY-ESO-1^{c259} TCR α chain (SEQ ID NO: 2). In another embodiment, the amino acid sequence of the NY-ESO-1 TCR comprises an amino acid sequence having at least 80%, 85%, 90%, 95%, 96%, 97%, 98%, 99%, or 100% sequence identity to NY-ESO-1^{c259} TCR β chain (SEQ ID NO: 3). In a further embodiment, the amino acid sequence of the NY-ESO-1 TCR α chain comprises an amino acid sequence having at least 80%, 85%, 90%, 95%, 96%, 97%, 98%, 99%, or 100% sequence identity to NY-ESO-1^{c259} TCR α chain (SEQ ID NO: 2), and the amino acid sequence of the NY-ESO-1 TCR β chain comprises an amino acid sequence having at least 80%, 85%, 90%, 95%, 96%, 97%, 98%, 99%, or 100% sequence identity to NY-ESO-1^{c259} TCR β chain (SEQ ID NO: 3). It will be appreciated that the percent sequence identity of each TCR chain (TCR α chain and TCR β chain) are not necessarily linked, and may vary from TCR α chain to TCR β chain.

The amino acid sequence of the NY-ESO-1^{c259} TCR α chain is shown in SEQ ID NO: 2.

(SEQ ID NO: 2)
METLLGLLILWLQLQWVSSKQEVTQIPAALSVPEGENLVLNCSFTDSAIY
NLQWFRQDPGKGLTSLLLIQSSQREQTSGRLNASLDKSSGRSTLYIAASQ
PGDSATYLCAVRPLYGGSYIPTFGRGTSLIVHPYIQNPDPAVYQLRDSKS
SDKSVCLFTDFDSQTNVSQSKDSDVYITDKTVLDMRSMDFKSNSAVAWSN
KSDFACANAFNNSIIPEDTFFPSPESSCDVKLVEKSFETDTNLNFQNLSV
IGFRILLLKVAGFNLLMTLRLWSS.

The amino acid sequence of the NY-ESO-1^{c259} TCR β chain is shown in SEQ ID NO: 3.

(SEQ ID NO: 3)
RMSIGLLCCAALSLLWAGPVNAGVTQTPKFQVLKTGQSMTLQCAQDMNHE
YMSWYRQDPGMGLRLIHYSVGAGITDQGEVPNGYNVSRSTTEDFPLRLLS
AAPSQTSVYFCASSYVGNTGELFFGEGSRLTVLEDLKNVFPPEVAVFEPS
EAEISHTQKATLVCLATGFYPDHVELSWWVNGKEVHSGVSTDPQPLKEQP
ALNDSRYCLSSRLRVSATFWQNPRNHFRCQVQFYGLSENDEVVTQDRAKP
VTQIVSAEAWGRADCGFTSESYQQGVLSATILYEILLGKATLYAVLVSAL
VLMAMVKRKDSRG.

The present invention also provides a nucleic acid construct comprising a first nucleic acid sequence encoding a CD8 co-receptor or fragment thereof, and a second nucleic acid sequence encoding a T cell receptor (TCR). In one embodiment, the CD8 co-receptor is CD8α. In another embodiment, the CD8 co-receptor comprises a nucleic acid sequence having at least 80%, 85%, 90%, 95%, 96%, 97%, 98%, 99%, or 100% sequence identity to CD8α (SEQ ID NO: 4).

The nucleic acid sequence of CD8α is shown in SEQ ID NO: 4.

(SEQ ID NO: 4)
ATGGCCTTACCAGTGACCGCCTTGCTCCTGCCGCTGGCCTTGCTGCTCCA
CGCCGCCAGGCCGAGCCAGTTCCGGGTGTCGCCGCTGGATCGGACCTGGA
ACCTGGGCGAGACAGTGGAGCTGAAGTGCCAGGTGCTGCTGTCCAACCCG
ACGTCGGGCTGCTCGTGGCTCTTCCAGCCGCGCGGCGCCGCCGCCAGTCC
CACCTTCCTCCTATACCTCTCCCAAAACAAGCCCAAGGCGGCCGAGGGGC
TGGACACCCAGCGGTTCTCGGGCAAGAGGTTGGGGGACACCTTCGTCCTC
ACCCTGAGCGACTTCCGCCGAGAGAACGAGGGCTACTATTTCTGCTCGGC
CCTGAGCAACTCCATCATGTACTTCAGCCACTTCGTGCCGGTCTTCCTGC
CAGCGAAGCCCACCACGACGCCAGCGCCGCGACCACCAACACCGGCGCCC
ACCATCGCGTCGCAGCCCCTGTCCCTGCGCCCAGAGGCGTGCCGGCCAGC
GGCGGGGGGCGCAGTGCACACGAGGGGGCTGGACTTCGCCTGTGATATCT
ACATCTGGGCGCCCTTGGCCGGGACTTGTGGGGTCCTTCTCCTGTCACTG
GTTATCACCCTTTACTGCAACCACAGGAACCGAAGACGTGTTTGCAAATG
TCCCCGGCCTGTGGTCAAATCGGGAGACAAGCCCAGCCTTTCGGCGAGAT
ACGTCGGTTCAAGAGCTAAAAGAAGTGGTAGTGGTGCCCCTGTGA.

In another embodiment, the TCR in the nucleic acid construct is affinity matured. In another embodiment, the TCR comprises an α chain and a β chain. In another embodiment, the TCR is a NY-ESO-1 TCR. In another embodiment, the nucleic acid sequence of the NY-ESO-1 TCR comprises a nucleic acid sequence having at least 80%, 85%, 90%, 95%, 96%, 97%, 98%, 99%, or 100% sequence identity to NY-ESO-1^{c259} TCR a chain (SEQ ID NO: 5). In another embodiment, the nucleic acid sequence of the NY-ESO-1 TCR comprises a nucleic acid sequence having at least 80%, 85%, 90%, 95%, 96%, 97%, 98%, 99%, or 100% sequence identity to NY-ESO-1^{c259} TCR β chain (SEQ ID NO: 6). In a further embodiment, the nucleic acid sequence of the NY-ESO-1 TCR a chain comprises a nucleic acid sequence having at least 80%, 85%, 90%, 95%, 96%, 97%, 98%, 99%, or 100% sequence identity to NY-ESO-1^{c259} TCR α chain (SEQ ID NO: 5), and the nucleic acid sequence of the NY-ESO-1 TCR β chain comprises a nucleic acid sequence having at least 80%, 85%, 90%, 95%, 96%, 97%, 98%, 99%, or 100% sequence identity to NY-ESO-1^{c259} TCR β chain (SEQ ID NO: 6).

The nucleic acid sequence of NY-ESO-1^{c259} TCR α chain is shown in SEQ ID NO: 5.

(SEQ ID NO: 5)
ATGGAGACCCTGCTGGGCCTGCTGATCCTGTGGCTGCAGCTCCAGTGGGT
GTCCAGCAAGCAGGAGGTGACCCAGATCCCTGCCGCCCTGAGCGTGCCCG
AGGGCGAGAACCTGGTGCTGAACTGCAGCTTCACCGACTCCGCCATCTAC
AACCTGCAGTGGTTCCGGCAGGACCCCGGCAAGGGCCTGACCAGCCTGCT
GCTGATCCAGAGCAGCCAGCGGGAGCAGACCAGCGGACGGCTGAACGCCA
GCCTGGACAAGAGCAGCGGCCGGAGCACCCTGTACATCGCCGCCAGCCAG
CCCGGCGACAGCGCCACCTACCTGTGCGCTGTGCGGCCTCTGTACGGCGG
CAGCTACATCCCCACCTTCGGCAGAGGCACCAGCCTGATCGTGCACCCCT
ACATCCAGAACCCCGACCCCGCCGTGTACCAGCTGCGGGACAGCAAGAGC
AGCGACAAGTCTGTGTGCCTGTTCACCGACTTCGACAGCCAGACCAATGT
GAGCCAGAGCAAGGACAGCGACGTGTACATCACCGACAAGACCGTGCTGG
ACATGCGGAGCATGGACTTCAAGAGCAACAGCGCCGTGGCCTGGAGCAAC
AAGAGCGACTTCGCCTGCGCCAACGCCTTCAACAACAGCATTATCCCCGA
GGACACCTTCTTCCCCAGCCCCGAGAGCAGCTGCGACGTGAAACTGGTGG
AGAAGAGCTTCGAGACCGACACCAACCTGAACTTCCAGAACCTGAGCGTG
ATCGGCTTCAGAATCCTGCTGCTGAAGGTGGCCGGATTCAACCTGCTGAT
GACCCTGCGGCTGTGGAGCAGC.

The nucleic acid sequence of NY-ESO-1^{c259} TCR β chain is shown in SEQ ID NO: 6.

(SEQ ID NO: 6)
AGGATGAGCATCGGCCTGCTGTGCTGCGCCGCCCTGAGCCTGCTGTGGGC
AGGACCCGTGAACGCCGGAGTGACCCAGACCCCCAAGTTCCAGGTGCTGA
AAACCGGCCAGAGCATGACCCTGCAGTGCGCCCAGGACATGAACCACGAG
TACATGAGCTGGTATCGGCAGGACCCCGGCATGGGCCTGCGGCTGATCCA
CTACTCTGTGGGAGCCGGAATCACCGACCAGGGCGAGGTGCCCAACGGCT
ACAATGTGAGCCGGAGCACCACCGAGGACTTCCCCCTGCGGCTGCTGAGC
GCTGCCCCCAGCCAGACCAGCGTGTACTTCTGCGCCAGCAGCTATGTGGG
CAACACCGGCGAGCTGTTCTTCGGCGAGGGCTCCAGGCTGACCGTGCTGG
AGGACCTGAAGAACGTGTTCCCCCCCGAGGTGGCCGTGTTCGAGCCCAGC
GAGGCCGAGATCAGCCACACCCAGAAGGCCACACTGGTGTGTCTGGCCAC
CGGCTTCTACCCCGACCACGTGGAGCTGTCCTGGTGGGTGAACGGCAAGG
AGGTGCACAGCGGCGTGTCTACCGACCCCCAGCCCCTGAAGGAGCAGCCC
GCCCTGAACGACAGCCGGTACTGCCTGTCCTCCAGACTGAGAGTGAGCGC
CACCTTCTGGCAGAACCCCCGGAACCACTTCCGGTGCCAGGTGCAGTTCT
ACGGCCTGAGCGAGAACGACGAGTGGACCCAGGACCGGGCCAAGCCCGTG
ACCCAGATTGTGAGCGCCGAGGCCTGGGGCAGGGCCGACTGCGGCTTCAC
CAGCGAGAGCTACCAGCAGGGCGTGCTGAGCGCCACCATCCTGTACGAGA
TCCTGCTGGGCAAGGCCACCCTGTACGCCGTGCTGGTGTCTGCCCTGGTG
CTGATGGCTATGGTGAAGCGGAAGGACAGCCGGGGCTAA.

In one embodiment, expression vectors are provided comprising the nucleic acid construct of the present invention. In another embodiment, the nucleic acid construct may be introduced directly into T cells using gene editing techniques. In another embodiment, modified T cells comprising the nucleic acid constructs or expression vectors are provided.

Also provided herein are modified T cells for use in therapy. In one embodiment, the therapy is allogeneic. In another embodiment, the therapy is autologous.

Also provided herein are methods of engineering a modified T cell comprising (i) providing a T cell; (ii) introducing an expression vector comprising a nucleotide construct encoding a CD8 co-receptor or fragment thereof and a T cell receptor (TCR) of the present invention into said T cell; and (iii) expressing said CD8 co-receptor or fragment thereof and T cell receptor (TCR) in the modified T cell.

In one embodiment, pharmaceutical compositions comprising a plurality of modified T cells that present a CD8 co-receptor or fragment thereof and a TCR, and a pharmaceutically acceptable carrier are provided. In one embodiment, the pharmaceutical compositions comprise allogeneic T cells. In another embodiment, the pharmaceutical compositions comprise autologous T cells.

In another embodiment, methods of treating cancer in a human are provided comprising administering an effective amount, e.g., therapeutically effective amount of said pharmaceutical composition to said human. In one embodiment, the methods further comprise expanding a population of said modified T cells ex vivo prior to administering to said human. The quantity and frequency of administration will be determined by such factors as the condition of the patient, and the type and severity of the patient's disease, although appropriate dosages may be determined by clinical trials. In some embodiments, the cancer may be synovial sarcoma, non-small-cell lung carcinoma (NSCLC), myxoid round cell liposarcoma (MRCLS), or multiple myeloma (MM).

One of ordinary skill in the art would recognize that multiple administrations of the compositions contemplated herein may be required to effect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & sons, NY, N.Y.).

In another embodiment, the present invention also provides a population of modified T cells as described herein, a nucleic acid construct as described herein, a vector as described herein, or a pharmaceutical composition as described herein for use in a method of treating a subject afflicted with cancer.

All publications, including but not limited to patents and patent applications, cited in this specification are herein incorporated by reference as though fully set forth.

As used herein and in the claims, the singular forms “a,” “and,” and “the” include plural reference unless the context clearly dictates otherwise. Thus, for example, reference to “a peptide chain” is a reference to one or more peptide chains and includes equivalents thereof known to those skilled in the art.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein.

Concentrations described herein are determined at ambient temperature and pressure. This may be, for example, the temperature and pressure at room temperature or in within a particular portion of a process stream. Preferably, concentrations are determined at a standard state of 25° C. and 1 bar of pressure.

The term “about” means a value within two standard deviations of the mean for any particular measured value.

The term “activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The terms “adoptive cellular therapy” or “adoptive immunotherapy” as used herein, refer to the adoptive transfer of human T lymphocytes or NK lymphocytes that are engineered by gene transfer to express CARs or genetically modified TCRs, specific for surface antigens or peptide MHC complexes expressed on target cells. This can be used to treat a range of diseases depending upon the target chosen, e.g., tumor specific antigens to treat cancer. Adoptive cellular therapy involves removing a portion of a donor's or the patient's white blood cells using a process called leukapheresis. The T cells or NK cells may then be expanded and mixed with expression vectors comprising the CAR/TCR polynucleotide in order to transfer the CAR/TCR scaffold to the T cells or NK cells. The T cells or NK cells are expanded again and at the end of the expansion, the engineered T cells or NK cells are washed, concentrated, and then frozen to allow time for testing, shipping and storage until a patient is ready to receive the infusion of engineered cells.

“Affinity” is the strength of binding of one molecule to another. The binding affinity of an antigen binding protein to its target may be determined by equilibrium methods (e.g. enzyme-linked immunosorbent assay (ELISA) or radioimmunoassay (RIA)), or kinetics (e.g. BIACORE™ analysis).

The term “allogeneic” as used herein, refers to any material derived from a different animal of the same species.

The term “antigen” as used herein refers to a structure of a macromolecule which is selectively recognized by an antigen binding protein. Antigens include but are not limited to protein (with or without polysaccharides) or proteic composition comprising one or more T cell epitopes. As is contemplated herein, the target binding domains an antigen binding protein may recognize a sugar side chain of a glycoprotein rather than a specific amino acid sequence or of a macromolecule. Thus, the sugar moiety or sulfated sugar moiety serves as an antigen.

The term “anti-tumor effect” as used herein, refers to a biological effect which can be manifested by a reduction in the rate of tumor growth, decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “autologous” as used herein, refers to any material derived from a subject to which it is later to be re-introduced into the same subject.

The term “avidity” as used herein, is the sum total of the strength of binding of two molecules to one another at multiple sites, e.g. taking into account the valency of the interaction.

As used herein, the terms “cancer,” “neoplasm,” and “tumor” are used interchangeably and, in either the singular or plural form, refer to cells that have undergone a malignant transformation that makes them pathological to the host organism. Illustrative examples of cells that can be targeted by compositions and methods contemplated in particular embodiments include, but are not limited to the following cancers: synovial sarcoma, non-small-cell lung carcinoma (NSCLC), myxoid round cell liposarcoma (MRCLS), and multiple myeloma (MM). Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

The term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “cell immunotherapy” as used herein, refers to a type of therapy in which immunomodulatory cells are genetically modified in order to target disease and then introduced into the patient. Areas of key focus are introducing chimeric antigen receptors (CARs) or genetically modified T cell receptors (TCRs) onto immunomodulatory cells in order to make them target specific.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “domain” refers to a folded protein structure which retains its tertiary structure independent of the rest of the protein. Generally, domains are responsible for discrete functional properties of proteins and in many cases, may be added, removed or transferred to other proteins without loss of function of the remainder of the protein and/or of the domain.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein refers to that portion of the antigen that makes contact with a particular binding domain, e.g. the target binding domain of a TCR molecule. An epitope may be linear or conformational/discontinuous. A conformational or discontinuous epitope comprises amino acid residues that are separated by other sequences, i.e. not in a continuous sequence in the antigen's primary sequence. Although the residues may be from different regions of the peptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. As is contemplated herein the term epitope includes post-translational modification to a polypeptide that can be recognized by an antigen binding protein or domain, such as sugar moiety of a glycosylated protein.

The term “expression vector” as used herein, refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “immunomodulatory cell” as used herein, refers to a cell that functions in an immune response, or a progenitor or progeny thereof. Examples of immunomodulatory cells include: T cells (also known as T-lymphocytes) which may be inflammatory, cytotoxic, regulatory or helper T cells; B cells (or B-lymphocytes) which may be plasma or memory B-cells; natural killer cells; neutrophils; eosinophils; basophils; mast cells; dendritic cells; or macrophages.

The terms “individual,” “subject,” and “patient” are used herein interchangeably. In one embodiment, the subject is a mammal, such as a primate, for example a marmoset or monkey, or a human. In a further embodiment, the subject is a human.

The term “isolated” as used herein, means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “lentiviral vector” as used herein, means a vector derived from at least a portion of a lentivirus genome, including especially a self-inactivating lentiviral vector as provided in Milone et al., Mol. Ther. 17(8): 1453-1464 (2009). Other examples or lentivirus vectors that may be used in the clinic as an alternative to the pELPS vector, include but not limited to, e.g., the LentiVector® gene delivery technology from Oxford BioMedica, the LentiMax™ vector system from Lentigen and the like. Nonclinical types of lentiviral vectors are also available and would be known to one skilled in the art.

The term “lentivirus” as used herein, refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The term “promoter” as used herein is defined as a DNA sequence recognized by the synthetic machinery of the cell, or introduced synthetic machinery, required to initiate the specific transcription of a polynucleotide sequence. As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

“Sequence identity” as used herein is the degree of relatedness between two or more amino acid sequences, or two or more nucleic acid sequences, as determined by comparing the sequences. The comparison of sequences and determination of sequence identity may be accomplished using a mathematical algorithm; those skilled in the art will be aware of computer programs available to align two sequences and determine the percent identity between them. The skilled person will appreciate that different algorithms may yield slightly different results.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “specifically binds,” and grammatical variations thereof as used herein with respect to an antibody, is meant an antibody or antibody fragment which recognizes and binds with a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labelled “A” and the antibody, will reduce the amount of labelled A bound to the antibody.

The term “stimulation,” used in the context of immune-receptor engineered TCR/CAR T cells or CAR NK cells, is meant a primary response induced by binding of a stimulatory molecule (e.g., a TCR/CD3 or CAR/CD3 complex) with its cognate ligand thereby mediating a signal transduction event, such as, but not limited to, signal transduction via the CAR/CD3 or TCR/CD3 complex. Stimulation can mediate altered expression of certain molecules, such as downregulation of TGF-beta, and/or reorganization of cytoskeletal structures, and the like.

The term “T cell receptor” (“TCR”) as used herein, refers to the receptor present on the surface of T cells which recognizes fragments of antigen as peptides bound to major histocompatibility complex (MHC) molecules. Native TCRs exist in αβ and γδ forms, which are structurally similar but exist in different locations and are thought to have different functions. The extracellular portion of the TCR has two constant domains and two variable domains. The variable domains contain polymorphic loops which form the binding site of the TCR and are analogous to complementarity determining regions (CDRs) in antibodies. In the context of cell immunotherapies, the TCR is usually genetically modified to change or improve its antigen recognition. For example, WO01/055366 and WO2006/000830, which are herein incorporated by reference, describe retrovirus-based methods for transfecting T cells with heterologous TCRs. WO2005/113595, which is herein incorporated by reference, describes high affinity NY-ESO T cell receptors.

Suitable TCRs bind specifically to a major histocompatibility complex (MHC) on the surface of cancer cells that displays a peptide fragment of a tumor antigen. An MHC is a set of cell surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumor antigen i.e. an antigen which is present on a cancer cell but not the corresponding non-cancerous cell. T cells which recognise these peptide fragments may exert a cytotoxic effect on the cancer cell.

In some embodiments, the coding sequences for the individual chains of the TCR (e.g. TCR α and TCRβ chains) may be separated by a cleavage recognition sequence. This allows the chains of the TCR to be expressed as a single fusion which undergoes intracellular cleavage to generate the two separate proteins. Suitable cleavage recognition sequences are well known in the art and include 2A-furin sequence.

Preferably, the TCR is not naturally expressed by the T cells (i.e. the TCR is exogenous or heterologous). Heterologous TCRs may include αβ TCR heterodimers. Suitable heterologous TCRs may bind specifically to cancer cells that express a tumor antigen. For example, the T cells may be modified to express a heterologous TCR that binds specifically to MHCs displaying peptide fragments of a tumor antigen expressed by the cancer cells in a specific cancer patient. Tumor antigens expressed by cancer cells in the cancer patient may identified using standard techniques.

A heterologous TCR may be a synthetic or artificial TCR, i.e., a TCR that does not exist in nature. For example, a heterologous TCR may be engineered to increase its affinity or avidity for a tumor antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumor antigen expressed by cancer cells. Suitable methods of generated affinity enhanced TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901).

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transfer vector” as used herein, refers to a composition of matter which can be used to deliver an isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, polylysine compounds, liposomes, and the like. Examples of viral transfer vectors include, but are not limited to, adenoviral vectors, adeno-associated virus vectors, gamma retroviral vectors, lentiviral vectors, and the like.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

### EXAMPLES

### Example 1: Cloning Strategy and Results

A lentiviral vector transgene expression plasmid that encodes a single CD8α_F2A_NY-ESOc259α TCR_P2A_NY-ESOwtβ. TCR ORF was constructed. It was designed so that after transduction into T cells, the integrated vector transgene cassette would act as a template to produce each of the 3 individual proteins by means of translational peptide bond skipping at the end of each 2A moiety. In the design the residual C-terminal 2A moieties are then removed by Furin protease cleavage. The resultant CD8α protein forms a homodimer to aid the binding of class I peptide-HLA antigen by an affinity-enhanced NY-ESO α/β TCR.

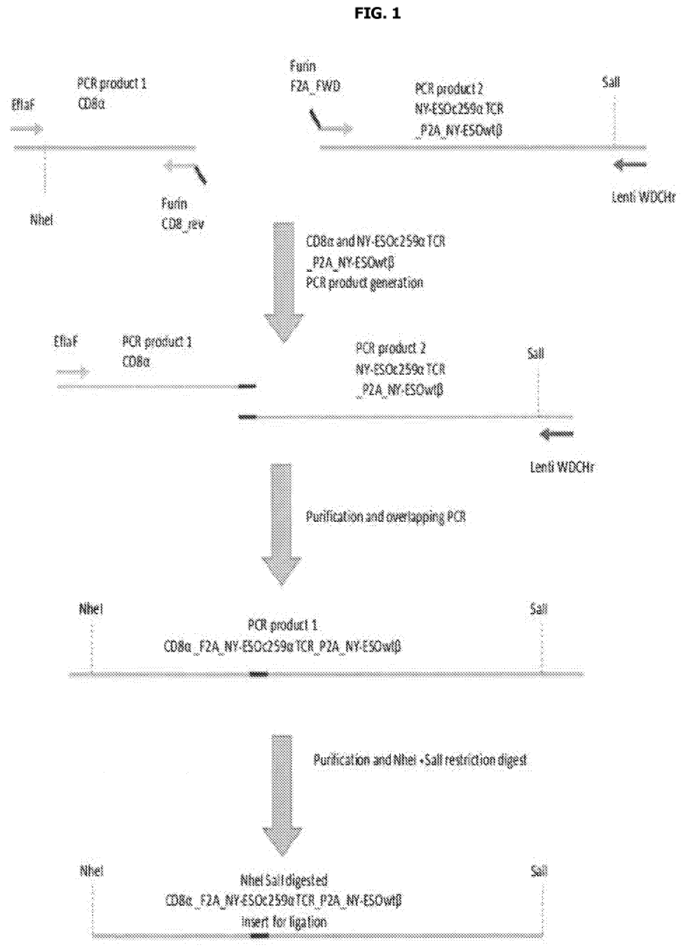

The full length sequence encoding CD8α_F2A_NY-ESOc259α TCR_P2A_NY-ESOwtβ was generated by the fusion of two individual PCR fragments by overlapping PCR. The first PCR fragment encodes CD8α with a C-terminal furin/SGSG linker sequence. The second PCR fragment encodes an N-terminal furin/SGSG linker sequence with the F2A skip peptide and the NY-ESOc259α TCR_P2A_NY-ESOwtβ TCR sequence. The nucleotide sequence encoding the furin/SGSG linker sequence provides the complementary region between the two PCR products. A schematic of the PCR strategy is shown in FIG. 1.

PCR product 1 was amplified from an existing in-house plasmid which encoded CD8α and F2A peptide in frame with an in-house TCR sequence. This fragment also contains a 5′ NheI site and Kozak sequence GCTAGCCGCCACC immediately upstream of the start ATG. PCR product 1 was amplified with the primers Lenti_eF1a (AGGCCAGCTTGGCACTTGAT) and Furin_CD8_rev (ACCACTACCACTTCTTTTAGCTCTTGAACCGACGTATCTCGCCGAAAGGC). Amplification with these primers produced a product of 871 bp (Note that the primer Lenti_eF1a is located in the EF1a promoter region and as such PCR product 1 encodes a partial fragment of the EF1a promoter which is removed following digest with NheI).

PCR product 2 was amplified from a separate in-house plasmid which encoded an additional gene fused to the NY-ESOc259α TCR_P2A_NY-ESOwtβ TCR sequence with an intervening F2A peptide. PCR product 2 was amplified with the primers FurinF2AF (GGTTCAAGAGCT AAAAGAAGTGGTAGTGGTGCCCCTGTGAAGCAGACC) and Lenti WDCHr (CGTATCCACATAGCGTAAAAGG). Amplification with these primers produced a product of 2038 bp. This fragment encodes the TCR sequence with a 3′ SalI GTCGAC site immediately following the TAA stop codon (Note that the primer Lenti WDCHr is located within the lenti backbone (WPRE sequence) and this additional sequence is removed following digest with SalI).

Following PCR, both products were purified by gel extraction and fused together by overlapping PCR with the 5′ primer Lenti_eF1a and the 3′ primer Lenti WDCHr. This amplification produced a product of 2879 bp. Following amplification of the full length CD8α_F2A_NY-ESOc259α TCR_P2A_NY-ESOwtβ sequence, the PCR product was gel purified and digested with NheI and SalI. The digested product was ligated into a lenti vector backbone between unique NheI and SalI sites. Clones from this ligation were screened by restriction enzyme digest and DNA sequencing. A single clone was selected for further purification of plasmid DNA on a mega prep scale.

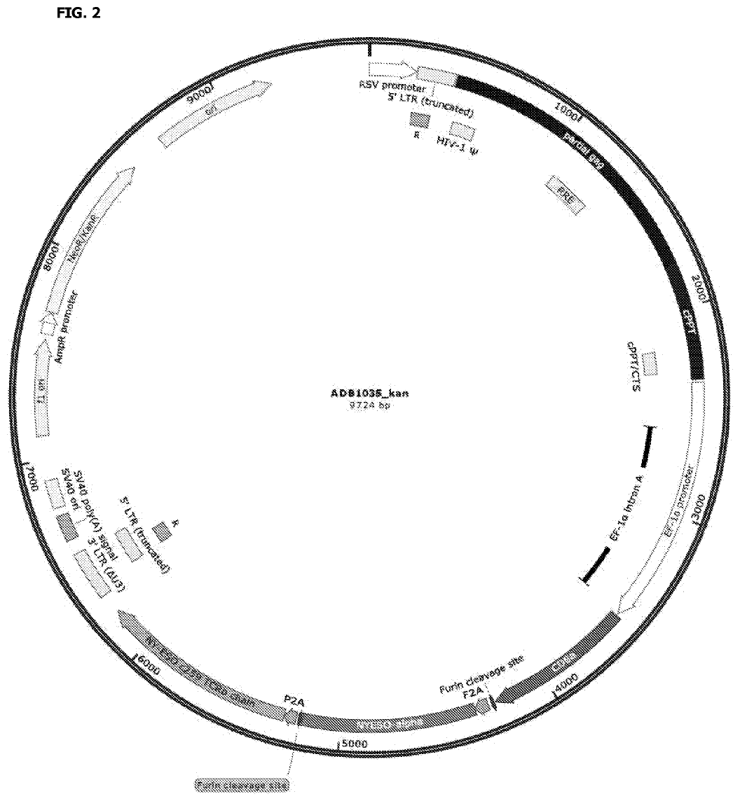

A new lenti vector backbone which had the WPRE sequence removed was generated in house. The CD8α_F2A_NY-ESOc259α TCR_P2A_NY-ESOwtβ coding sequence was removed from the construct generated above by restriction digest with NheI and SalI and sub-cloned to the new backbone between unique NheI and SalI restriction sites. This produced the lentivector ADB1035. The vector map of the variant, ADB1035_kan is presented in FIG. 2.

### Example 2: Impact of CD8α Expression on T Cell Activation

CD40 ligand (CD40L, also known as CD154) is primarily expressed on activated T cells, preferentially CD4+ T cells. It acts as a co-stimulatory molecule which binds CD40 on antigen presenting cells (APCs). CD40-CD40L interaction licences APCs to activate antigen specific naive CD8+ T cells. It is expressed in response to TCR-mediated signalling as well as non-physiological stimulation such as anti-CD3 targeting; and is transiently expressed (5 min post TCR activation to 6 hours).

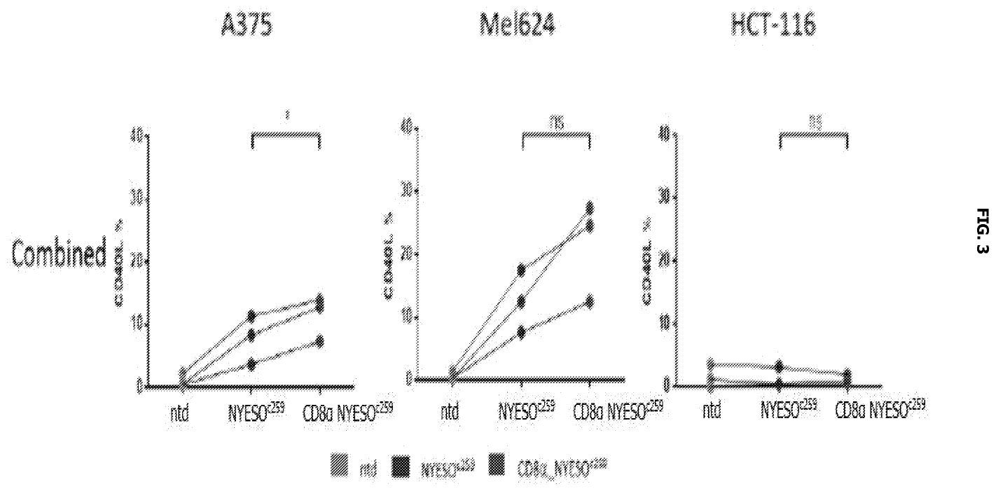

CD40L was used as a marker of early T cell activation in response to antigen, and measured in CD4+ T cells. Mock clinical scale Wave T cells from 3 donors (Wave 124, 147, 149) were incubated with target cells for 5 hours and stained for intracellular CD40L. Target cells used were A375 (NY-ESO-1+/LAGE-1A−), Mel624 (NY-ESO-1+/LAGE-1A+) and the negative control HCT-116 (NY-ESO-1/LAGE-1A−). Wells containing additional NY-ESO-1 peptide SLLMWITQC were included as positive controls and T cell alone (unstimulated) were included as a negative control reflecting CD40L baseline. Results are shown in FIG. 3. NY-ESO-1^{c259} T cells expressing the c259 TCR on the cell surface were identified by flow sorting cells using an antibody that recognises the NY-ESO-1^{c259} TCR with a small amount of cross-binding (approx. 5%) to endogenous TCR. Transduced cells are defined hereafter as Vbeta+. The data shows a consistent upregulation of CD40L by the NY-ESO-1^{c259} CD4+ T cells when presented with antigen positive targets cells (A375 and Mel624) compared to the nontransduced (ntd) T cells. This confirms that CD40L is upregulated in response to antigen and can be measured. The further response from CD8α NY-ESO-1^{c259} CD4+ T cells compared to the NY-ESO-1^{c259} T CD4+ T cells was highly variable, but there was a trend for enhanced CD40L expression due to CD8α co-expression. Because of the low sample number (n=2 or 3) there is limited statistical significance. When the data for each of the 3 waves was combined (by averaging the means), there was a modest but statistically significant difference between the response from CD8α NY-ESO-1^{c259} T compared with NY-ESO-1^{c259}T transduced CD4+ T cells when challenged with A375 cells (p=0.0232) but not with Mel624 (p=0.0979). Overall, the CD40L upregulation in response to antigen positive cell exposure suggests a trend of enhanced activation in the CD8α NY-ESO-1^{c259}CD4+ T cells over NY-ESO1^{c259} T CD4+ cells, although the low sample numbers limit statistical analysis.

### Example 3: Antigen-Specific T Cell Proliferation

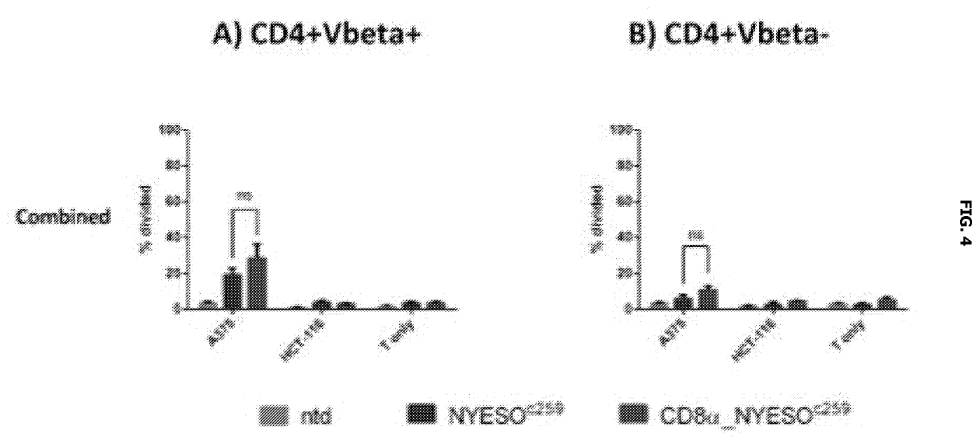

In order to determine the effect of CD8α homodimer co-receptor on CD8α NY-ESO-1^{c259} T cell proliferation, flow cytometry based proliferation assays of CD4+Vbeta+ and CD4+Vbeta− T cell subsets within ntd, NY-ESO^{c259} T, or CD8α NY-ESO^{c259} T cells in response to antigen positive (A375) and negative (HCT-116) cell lines were performed on 3 donors (waves 128, 147 and 149). Wave 128 was grown with no serum, whereas Waves 147 and 149 were grown with serum. A combined analysis of the data from these 3 donors for CD4+ T cells is shown in FIG. 4. The percentages of proliferating CD4+Vbeta+ (% divided; A) and CD4+Vbeta− (% divided; B) are shown as a mean±SEM across three donors: Wave147, Wave149 and Wave128 (combined). Cells were cultured for 3 days alone (T only) or co-cultured with antigen positive (A375) or antigen negative (HCT-116) cell lines. Statistical significance was assessed using paired two-tailed t-test. Proliferation of CD8+Vbeta− cells in response to antigen were observed in the same culture conditions. This is possibly a secondary effect induced by cytokines released from proliferating Vbeta+ T cells, rather than being antigen-driven.

CD4+ T cells do not normally recognise peptide in association with MHC-class I molecules. However, due to the high affinity of the NY-ESO-1^{c259} TCR, CD4+ T cells are no longer fully reliant on co-receptor ligation for their activation. Consistently across all three donors tested, there is a trend for enhanced proliferation of CD4+ transduced T cells in response to antigen when transduced with vector encoding both CD8α and NY-ESO-1^{c259} TCR.

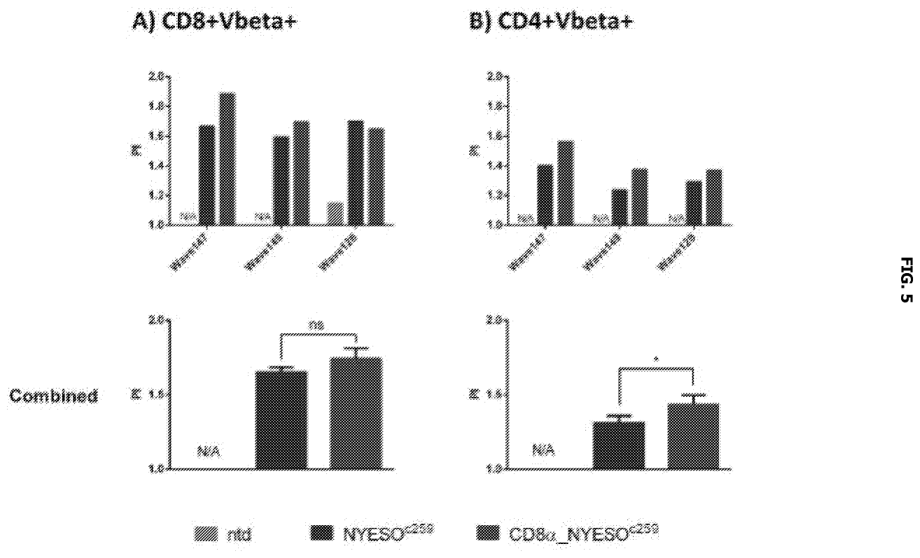

Antigen-specific proliferation was also assessed by calculating the proliferation index (PI) of the Vbeta+CD8+ and CD4+ T cell subsets in response to the NY-ESO-1 positive cell line A375 (FIG. 5). The PI accounts for the average number of divisions for all responding cells.

In this assay, cells loaded with a violet proliferation dye (VPD450) distribute that dye evenly between daughter cells. Reduction of the dye in a flow cytometry assay indicates cell division and thus proliferation. Rested, T cells were stained with VPD450 and incubated alone or in co-culture with antigen presenting cells (ratio T cells:target cells=5:1) and irradiated target cells in the presence or absence of 10⁻⁵ M NY-ESO-1 peptide SLLMWITQC for 3 days. A PI is calculated as the total number of divisions divided by the number of cells that went into division. The PI only takes into account the cells that underwent at least one division so only responding cells are reflected in the PI.

The PI was calculated for ntd, NY-ESO-1^{c259} T and CD8α NY-ESO-1^{c259} T cells from three different donors (Wave128, Wave147 and Wave149) (FIG. 5) and a combined analysis of the data was conducted by averaging the PI of the three T cell donor waves (FIG. 5, lower panel).

As can be seen from FIG. 5, while not statistically significant differences in PI were observed for CD8+ T cells, CD4+Vbeta+ T cells proliferated to a greater extent in CD8α NY-ESO-1^{c259} T than in NY-ESO-1^{c259} T cells in the combined analysis (p<0.05). While wave-scale showed significance in a combined analysis of waves, research scale proliferation data did not show consistent or significant increased proliferation of CD8a NY-ESO-1^{c259} T cells over NY-ESO-1^{c259} T cells. Although there were some differences in the protocols when comparing research scale to wavescale, it is not clear why there was a difference in results between the two methods.

### Example 4: The Effect of CD8α Expression on CD4+ Cell Th1 and Th2 Cytokine Responses

Naïve CD4+ T cells undergo polarisation to distinct subsets after activation which secrete different cytokine combinations. The first defined and best characterised of these subsets are the Th1 and Th2 subsets. Th1 cells are characterised by the secretion of cytokines such as IFN-γ, TNFα and IL2. They are thought to be mainly responsible for immune responses against intracellular pathogens by either enhancing CD8+ T cell responses or by directly activating macrophages to phagocytose intracellular pathogens. In contrast, Th2 cells typically secret the signature cytokines IL4, IL5 and IL13 which are thought to be important for humoral immunity by supporting B cell proliferation and differentiation and antibody class switching (Kim and Cantor, Cancer Immunol Res. 2014 February; 2(2):91-8).

Th1 cells are thought to have more potent anti-tumor effects than Th2 cells which may be attributed to the production of large amounts of IFN-γ that enhance the priming and expansion of CD8+ T cells. Furthermore, Th1 cells help recruit other immune cells including natural killer (NK) cells and type I macrophages to tumor sites which may act together to eradicate the tumors. Th1 cells and the cytokines they produce such as IFN-γ are strongly associated with good clinical outcome for many cancer types (Fridman et al., Nat Rev Cancer. 2012 Mar. 15; 12(4):298-306). In contrast, there is evidence to suggest that Th2 cells may instead promote tumor growth in some cancers (Kim and Cantor), and the majority of the time are associated with poor clinical outcome and aggressive tumors (Fridman et al.). Therefore, the induction or enhancement of Th1 cytokines by CD4+ T cells transduced with CD8α could be considered desirable within the tumor microenvironment, whereas a skewing towards a Th2 phenotype may be less favorable.

Changes in secretion of a panel of 25 cytokines and chemokines were measured using the Luminex™ Magpix® system when NY-ESO-1^{c259} T TCR or CD8α NY-ESO-1^{c259} T unseparated PBLs or CD4+ only fractions were challenged with NY-ESO-1 antigen. Cells were normalised for Vbeta transduction and incubated either with increasing concentrations of antigenic NY-ESO-1 peptide presented by T2 cells (panel A in each figure) or the antigen positive A375 cell line (panel B in each figure). T2 cells are deficient in a peptide transporter involved in antigen processing (TAP) and therefore fail to display endogenous MHC-peptide complexes. Supernatants were harvested after 24 and 48 hours. Selected Th1 (IL-2, GM-CSF, IFN-γ, TNFα) and Th2 cytokines (IL-4, IL-5, IL-10 and IL-13) known to be associated with CD4+ helper functions are discussed.

### Th1 Cytokine Response—Preclinical Wave Scale Data

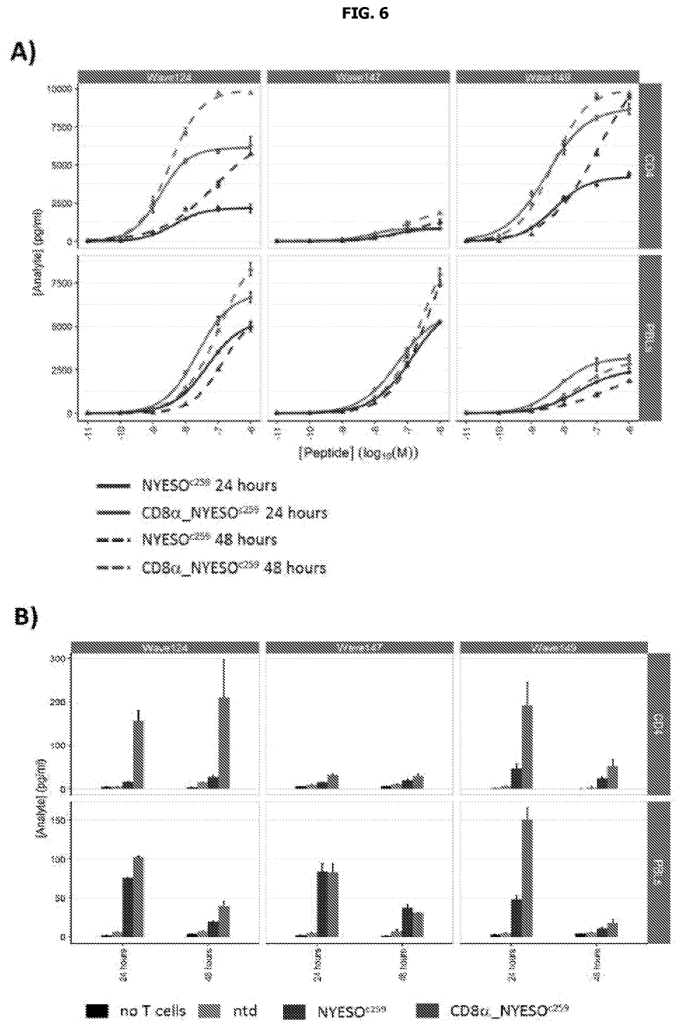

Interleukin-2 (IL-2) is a growth, survival and differentiation factor for T lymphocytes that plays a critical role in both promoting and controlling T cell responses and functions. IL-2 is produced mainly by CD4+ T cells early after activation and can act in either an autocrine or paracrine manner. It stimulates the survival, proliferation and differentiation of CD4+ and CD8+ T cells. FIG. 6 shows IL-2 release analysis by Luminex™ MAGPIX® assay with individual panels plotted for each donor (Wave124 (ACL118, ACL120), Wave147 (ACL112, ACL119), Wave149 (ACL111, ACL114) and unseparated (PBLs) or CD4 enriched (CD4) T cells. Upon stimulation with NY-ESO-1 peptide, both transduced unseparated T cells and CD4(+)-enriched fractions exhibited dose-dependent release of IL-2. For 2 of 3 donors examined, CD8α NY-ESO-1^{c259} T cells responded at a lower concentration of peptide in relation to NY-ESO-1^{c259} T cells. This higher sensitivity of the CD8α NY-ESO-1^{c259} T cells is reflected by a log shift of EC50 values at the 48 hour time point and suggests that CD8α NY-ESO-1^{c259} T cells may be more efficacious when engaging antigen low cells. Wave 147 did not respond in the same way, likely due to donor variation.

Interferon-gamma (IFN-γ) is produced by activated CD4+ and CD8+ T cells (but mainly by the activated CD8+ T cells) and NK cells. IFN-γ promotes the presentation of antigen to T cells by stimulating the expression of MHC molecules and many of the proteins involved in antigen processing. It also amplifies these actions by promoting the differentiation of CD4+ T cells to the IFN-γ producing Th1 subset and inhibiting the development of Th2 and Th17 cells. It is also the principal macrophage-activating cytokine.

Tumor Necrosis Factor alpha (TNFα) is a pro-inflammatory cytokine secreted in response to many different microbial products; mainly by tissue macrophages and dendritic cells, but also other cell types including adipocytes, CD4 T cells and fibroblasts. It enhances the adhesiveness of vascular endothelium for leukocytes and promotes trans-endothelial migration. It also synergises with IFN-γ in many of its actions, including MHC induction and macrophage activation. TNFα is an essential factor in mediating the immune response against bacteria and other infectious microbes and is cytotoxic to a wide variety of tumor cells.

Granulocyte-macrophage colony-stimulating factor (GM-CSF) has recently been tested as neoadjuvant in prostate cancer vaccine trial and proved to enhance recruitment of CD8+ cytotoxic T cells to tumor microenvironment. GM-CSF has been shown to preferentially enhance both the numbers and activity of type 1 dendritic cells (DC1), the DC subset responsible for initiating cytotoxic immune responses.

Similar results to IL2 and were seen for 2 of 3 donors for the Th1 cytokines IFN-γ, TNFα and GM-CSF, where the amount of these cytokines was greatly elevated, often several-fold, for CD8α NY-ESO-1^{c259} T in comparison with NY-ESO-1^{c259} TCR alone at the 48 hour timepoints. Correspondingly, the equivalent analyte response was achieved with a lower peptide concentration by CD8α NY-ESO-1^{c259} T when compared with NY-ESO-1^{c259} T, although these changes were not reflected in the same shift in EC₅₀ values as seen for IL2. It should be noted that these conclusions are based on only 2 donors, again wave 147 was an outlier, so no statistical analysis could be performed.

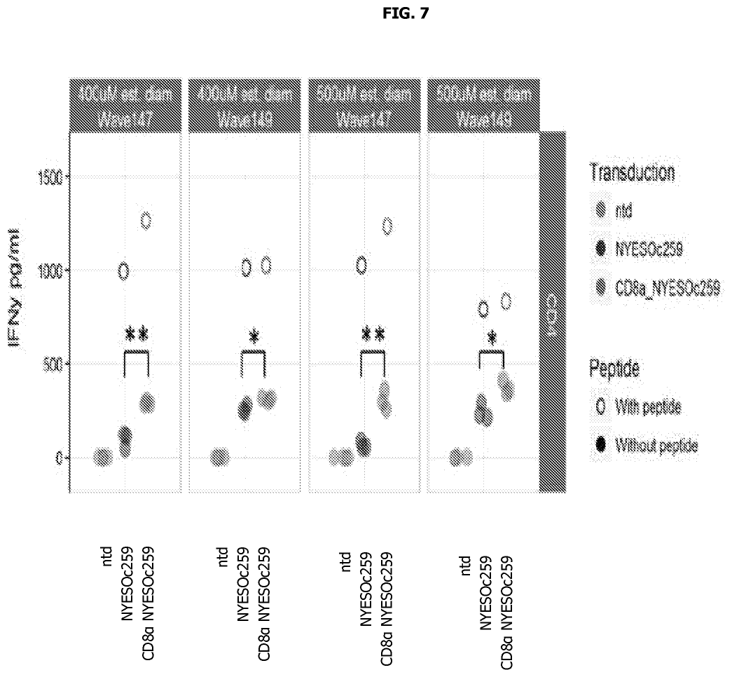

In addition to 2D assays, the response of T cells to antigen positive 3D spheroids was determined by measuring IFN-γ (FIG. 7). Supernatants were collected at 139h post T cell addition and the levels of IFN-γ in the supernatants were measured by ELISA. Graphs display levels of cytokine produced by peripheral blood lymphocytes (PBL), CD4+ or NY-ESO-1^{c259} T, CD8α NY-ESO-1^{c259} T cells or nontransduced (ntd) T cells incubated with A375-GFP 3D spheroids, with (open symbols) or without (filled symbols) 10 μM NY-ESO-1 SLLMWITQC peptide. Individual replicates are shown. All conditions without peptide are in triplicate, or single replicates with peptide. Two-tailed unpaired t-tests were performed comparing IFN-γ release by CD8α NY-ESO-1^{c259} T and CD8α NY-ESO-1^{c259} T cells for all fractions and donors without peptide (df=4 for all, ns=not significant, *=p<0.05, **=p<0.01). These data show a greater IFN-γ response by CD8α NY-ESO-1^{c259} T cells for both waves 147 and 149 in response to both medium and large scale 3D spheroids A375-GFP 3D spheroids (p<0.05 or p<0.01).

### Th2 Cytokine Response

Th2 CD4+ T cells are regarded as inhibitory with respect to the adoptive immune response and have been associated with poor cancer prognosis. The most widely described Th2 cytokines IL4, 1L5, 11_10 and IL13 were examined in this study.

At research scale, there was no significant production of Th2 cytokines from either CD8α NY-ESO-1^{c259} T cells or NY-ESO-1^{c259} T cells. At pre-clinical scale, in general, Th2 cytokine release by TCR-transduced CD4+ T cells in response to exogenous NY-ESO-1 peptide using T2 cells was close to the detection limit of the assay. Only Wave149 CD8a NY-ESO-1^{c259} T CD4+ T cells secreted substantial amounts of IL-4 and IL-13 (data not shown). Overall, the Th2 cytokine response to NY-ESO-1 peptide seemed to be very donor dependent and may depend on the inherent Th1/Th2 balance present in each donor. When challenged with endogenous peptide-MHC class I complex, CD4+ T cells generally gave background levels of cytokines. A hint of NY-ESO-1 directed response could be observed with IL-4 secretion, but differences between CD8α NY-ESO-1^{c259} T and NY-ESO-1 were minimal.

### Example 5: The Effect of CD8α Expression on NY-ESO-1^{c259} CD4+ T Cell Release of Other Cytokines and Chemokines

In addition to Th1 and Th2 responses, the levels of additional cytokines and chemokines were examined. The key differences between NY-ESO-1^{c259} T cells and CD8α NY-ESO-1^{c259} T cells are summarized in Table 1.

TABLE 1
Summary of LuminexTM MAGPIX Assay Data for Cytokines/Chemokines
Differentially Secreted by CD8α NY-ES0-1^{c259} and CD8α NY-ES0-1^{c259} Transduced T Cells

| | | | NY-ESO^{c259} | CD8α_NY- |
|---|---|---|---|---|
| | | | CD4+ T cells | ESOc259 CD4+T |
| Cytokine / | | Selected Roles in immune | (Analyte ± | cells (Analyte ± |
| Chemokine | Principal Cell Source | Response | SEM) (pg/ml) | SEM) (pg/ml) |
| IFNα | Plasmacytoid | Anti-viral state, increased | 70 ± 24 | 112 ± 23 |
| | dendritic cells, | class \| MHC expression, | | |
| | macrophages | activation of NK cells | | |
| IL-2R | T cells | T cells: inhibits IL-2- | 93 ± 28 | 157 ± 46 |
| | | mediated proliferation | | |
| IL-12 | Macrophages, | T cells: Th1 differentiation | 24 ± 6 | 41 ± 6 |
| | dendritic cells | NK cells and T cells: IFNγ | | |
| | | synthesis, increased | | |
| | | cytotoxic activity | | |
| MIG | Monocytes, | Effector T cell recruitment | 32 ± 22 | 132 ± 112 |
| | macrophages, and | | | |
| | endothelial cells | | | |
| P-10 | Endothelium, mast | Effector T cell recruitment | 185 ± 153 | 655 ± 444 |
| | cells, leukocytes, | | | |
| | tissue cells | | | |
| RANTES | Endothelium., mast | Mixed leukocyte | 46 ± 12 | 125 ± 69 |
| | cells, leukocytes, | recruitment | | |
| | tissue cells | | | |
| MP-1α | Endothelium, mast | Mixed leukocyte | 148 ± 40 | 309 ± 127 |
| | cells, leukocytes, | recruitment | | |
| | tissue cells | | | |
| MP-1β | Endothelium, mast | T cell, dendritic cell, | 126 ± 44 | 254 ± 89 |
| | cells, leukocytes, | monocyte, and NK cell | | |
| | tissue cells | recruitment | | |

From the results in Table 1, it was observed that CD4+CD8α NY-ESO-1^{c259} T cells secrete many chemokines and cytokines that mediate effector T cell recruitment, with a trend for elevated levels in comparison with CD4+NY-ESO-1^{c259} T cells. These include IFNα, shown to upregulate HLA class 1 in cancer; the chemokine IP-10, thought to play an important role in recruiting activated T cells and a is a potent inhibitor of angiogenesis in mice; and RANTES, a potent chemoattractant for many cell types including NK cells and memory T cells. These results could indicate that co-expression of CD8α may result in increased trafficking of T cells and additional anti-tumor effects.

### Example 6: Enhancement of Granzyme B Expression by CD8α Co-Expression

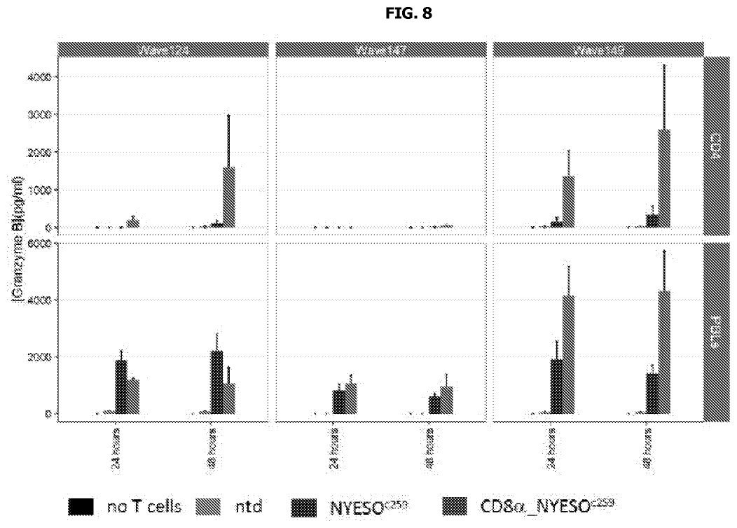

Granzyme B is a serine protease found in the granules of CTLs. It is released by T cells and uptake results in an apoptotic cascade and killing of target cells. As such its expression is a surrogate for T cell killing activity. The cytotoxic function of the transduced T cells was assessed via Granzyme B ELISAs in the supernatants collected from the 24 hour and 48 hour co-culture assays (Th1/Th2 cytokine response) co-cultured with A375 cells (FIG. 8). When challenged with antigen positive A375 cells there is an overall trend for more granzyme B to be secreted from the CD8α NY-ESO-1^{c259} T over the NY-ESO-1c259 T cells, especially the CD4+ isolated cells from Waves124 and 149. The differences are small, however killing through granzyme B is a minor function for CD4+ T cells so even small differences are notable. The trend supports the proposed function of the CD8a co-receptor in helping TCR-transduced CD4+ T cells respond better to antigen presented on class I MHC complex.

An NY-ESO-1 peptide dilution assay measuring Granzyme B was also performed. At higher peptide concentrations, this showed a trend towards a greater response from CD8α NY-ESO-1^{c259} T as compared with NY-ESO-1^{c259} T.

### Granzyme B Expression in a 3-Dimensional (3D) Cell Culture Assay

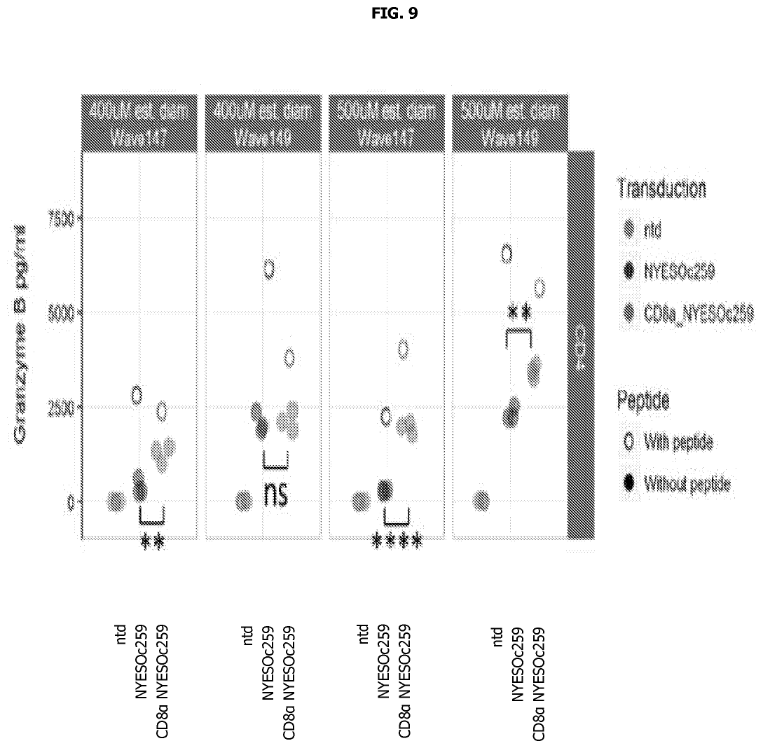

A number of assays (including granzyme B and cytotoxicity) were conducted in a 3D spheroid system. A375 human melanoma cells transduced with vector encoding GFP (A375-GFP) were grown in plates with a cell-repellent coating, to facilitate adhesion of cells to one another to form the 3D cell structures. Cells were seeded at two different densities to produce “medium” (400 μm diameter) and “large” (500 μm diameter) structures. Wavescale T cells normalised for transduction efficiency were then added. For this assay the 2 waves, 147 and 149 were tested. Results for the granzyme B assay are shown in FIG. 9. Supernatants were collected at 139h post T cell addition and the levels of Granzyme B in the supernatants were measured by ELISA. The graphs in FIG. 9 display levels of cytokine produced by peripheral blood lymphocytes (PBL), CD4+ or CD8+ NY-ESO-1^{c259} T cells, CD8α NY-ESO-1^{c259} T cells, or ntd T cells incubated with A375-GFP 3D microtissues, with or without 10 μM NY-ESO-1 SLLMWITQC peptide. Individual replicates are shown. All conditions without peptide are in triplicate, or single replicates with peptide. Two-tailed unpaired t-tests were performed. FIG. 9 shows that for both waves, CD8α NY-ESO-1^{c259} T cells produce more granzyme B than NY-ESO-1^{c259} T cells with the results reaching statistical significance. For wave 147 this was seen at both sizes of 3D cell structures: 400 μM (P<0.01) and 500 μM (p<0.0001), for wave 149 only at the 500 μM size (p<0.01).

### Example 7: Cytotoxicity of CD8α T Cells

Three sets of cytotoxic T cell killing assays were conducted comparing CD8α NY-ESO-1^{c259} T cells to NY-ESO-1^{c259} T cells: research scale, pre-clinical wave scale in 2D cell cultures and in 3D cell culture killing assays.

### Research Scale Cytotoxicity of CD8a

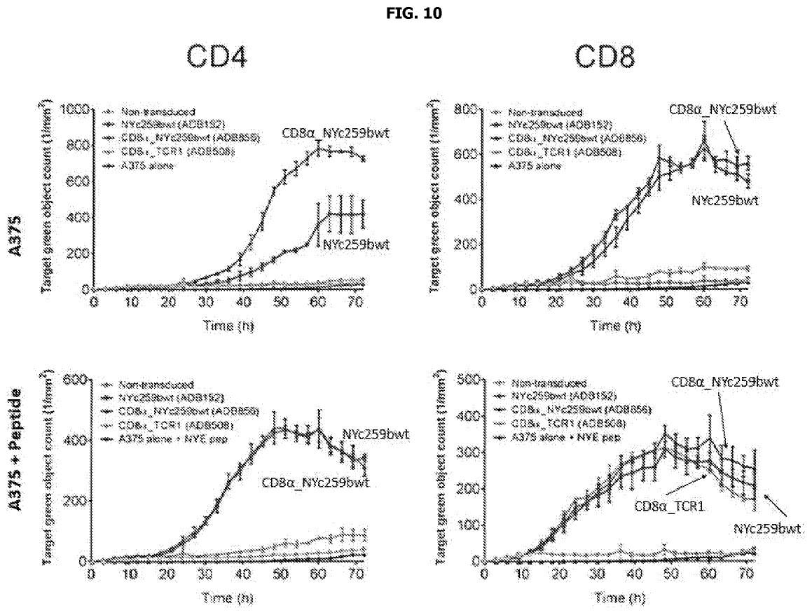

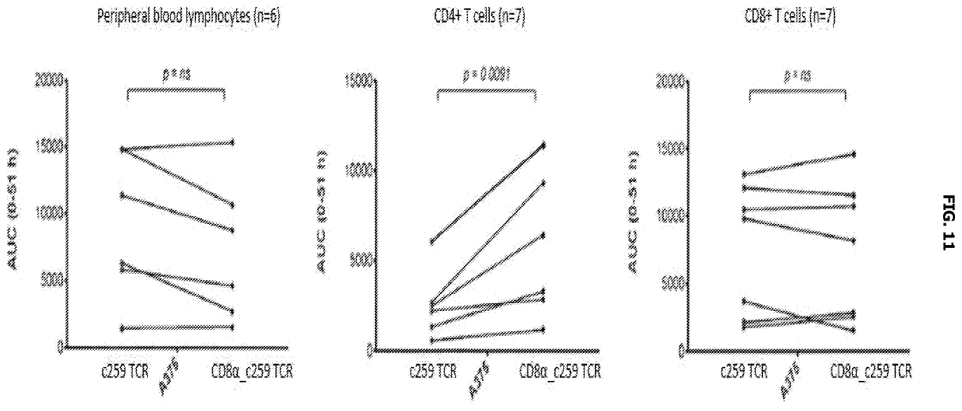

At research scale, PBLs, CD4+ and CD8+ cells from 7 donors were separated from whole blood, transduced, expanded for 14 days before being assayed. A mock TCR (TCR1), with no affinity for NY-ESO-1 was used as a control. HLA-A2+/NY-ESO-1+ human melanoma cell lines A375, SKMel37 and NY-ESO-1 antigen negative cells HepG2 were used as target cells. Cells were pulsed with SLLMWITQV or TC1 peptide. Transduction efficiencies were normalised by addition of non-transduced T cells from the same donor. Effector T cell killing was measured using CellPlayer™ 96-Well Kinetic Caspase-3/7 reagent (Essen Biosciences) with images acquired on IncyCyte Zoom system. Data images were acquired every two hours following the addition of T cells, for up to 96 hours. Images were analysed, including an exclusion gate to eliminate dead/dying effector cells from the analysis. The area under the curve (AUC) measurements of the cytotoxicity activity of CD8α NY-ESO-1^{c259} T cells compared with NY-ESO-1^{c259} T cells against A375 target cells followed the assumption that for all donors analysed, peak killing had occurred at 51 hr, so the 0-51 hr AUC was used. FIG. 10 shows a representative curve for one donor where the target cell was A375 and FIG. 11 shows an overall collective AUC analysis for the 7 donors where the target cell was A375.

For all seven donors tested PBL and CD8+ T cells transduced with c259 TCR alone were able to robustly kill A375 cells expressing NY-ESO-1. CD4+NY-ESO-1^{c259} T cells demonstrated reduced levels or no killing against the same target cells, however CD8α NY-ESO-1^{c259} T cells showed significant improvement in their ability to kill A375 cells (FIG. 11). The faster killing kinetics of CD8+ T cells may have masked any improvement in killing when CD8α was co-expressed with NY-ESO-1^{c259} TCR in the CD8 fraction and in PBLs (FIGS. 8 and 9). When the A375 targets were pulsed with NY-ESO peptide, killing by CD4+ T cells transduced with c259 TCR alone or CD8α c259 was comparable.

The data suggest that the co-expression of CD8α with c259 TCR in CD4 T cells has enhanced their peptide sensitivity by increasing binding avidity of the TCR-pMHC interaction. This could be important when levels of antigen are low as no differences were observed between NY-ESO-1^{c259} T cells and CD8α NY-ESO-1^{c259} T cells when the target cells were pulsed with high levels of cognate peptide (see FIG. 8). In addition, the improvement in killing is TCR driven as no non-specific killing was observed against antigen negative targets or when CD8α was co-expressed with an irrelevant TCR1 in T cells (FIG. 11).

For the cell line SKMel37, similar results were seen with a trend for increased killing by CD8α NY-ESO-1^{c259}CD4+ cells compared to CD4+NY-ESO-1^{c259} cells in 5 out of 7 donors, although no formal statistical analysis of the data was done. Kinetics of killing were slower than for A375, perhaps as a result of lower antigen levels.

### Pre-Clinical Wave Scale Cytotoxicity Data

To further assess the cytotoxicity of CD8α NY-ESO-1^{c259} T cells compared with NY-ESO-1^{c259} T cells, IncuCyte killing assays were performed with antigen positive cell lines A375, NCI-H1755, Mel624 and negative controls lines Colo205.A2, Caski.A2 and HCT-116. These assays were carried out using T cells grown at wave scale (2 litre culture bags) to better mimic cell manufacture for clinical trials.

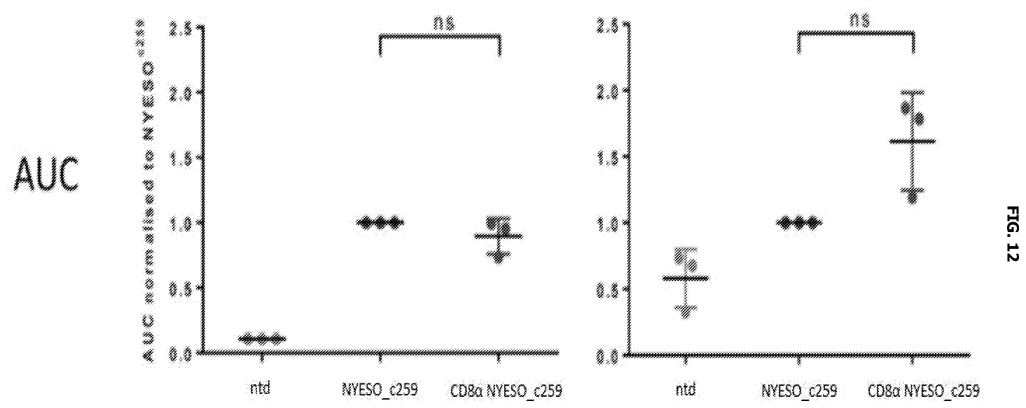

Target cells were incubated with isolated CD4+ T cells, alongside PBLs. CD8α NY-ESO-1^{c259} T cells and NY-ESO-1^{c259} T cells were also normalized for transduction efficiency (total Vbeta+) prior to each assay and prior to cell separation. Additional samples with NY-ESO-1 SLLMWITQC peptide were included in each assay to control for the ability of target cells to present antigen and for T cell functionality. FIG. 12 shows the results for one of the antigen positive cell lines, Mel624 assayed. Mel624 cells were seeded to each well of a 384 well-format plate. T cells were either the unseparated Wave product (PBLs) or the CD4+ enriched fraction. Images were taken on an IncuCyte Zoom every 2 hours for a period of 96 hours. The panels in FIG. 12 shows area under the curve (AUC) expressed as a ratio compared to NY-ESO-1^{c259} T cell response for all assays (mean AUC for both Wave149 assays combined with data from Wave124 and Wave147) and calculated at 72 h, which represents the time target cells treated with ntd T cells start dying off due to over confluency or nutrient deprivation. Each point represents one assay/Wave T cell. Statistical significance was assessed by a paired t-test.

FIG. 12 shows a trend for increased killing by the CD4+ fraction of CD8α NY-ESO-1^{c259} T versus NY-ESO-1^{c259} T cells which did not reach statistical significance. Very similar results were obtained for the other antigen positive cell lines A375 and NCI-1755 (data not shown).

Differences existed in the culture conditions for the pre-clinical wave bag T cell production, compared to those used for the research scale package of experiments. This may explain some of the differences between the research and the preclinical experiments in the effect of CD8α on cell killing; e.g. perhaps differences in the time of removal for the CD3/CD28 beads between the research and preclinical T cell processes led to differential T cell activation states in vitro in culture.

### Preclinical Wave Scale 3D Spheroid Cytotoxicity Assay

NY-ESO-1 expressing, HLA-A*02 positive A375-GFP cells were grown in plates with a cell-repellent coating, to facilitate adhesion of cells to one another to form a 3D cell spheroid. Cells were seeded at two different densities to produce “medium” (400 μm diameter) and “large” (500 μm diameter) 3D “cell structures”. Wavescale T cells were normalized for transduction efficiency before addition to the assays.

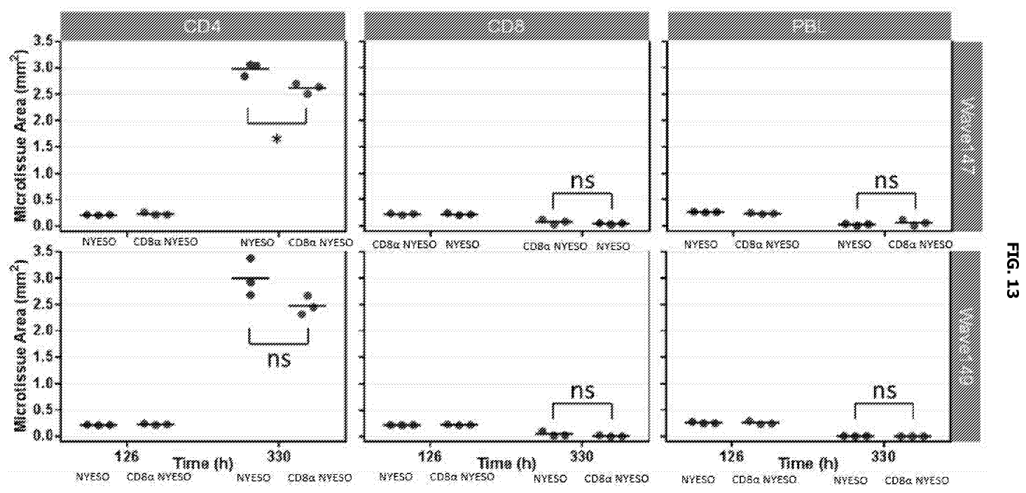

Across medium and large spheroids, the CD8α NY-ESO-1^{c259} T cells showed a trend for improved killing as compared with NY-ESO-1^{c259} T cells in the CD4+ T cell fraction (FIG. 13). Imaging was carried out every 3 hours using the IncuCyte ZOOM. The plots in FIG. 13 show the core fluorescence area of each 3D spheroid with Wave147 and Wave149 NY-ESO-1^{c259} T or CD8α NY-ESO-1^{c259} T cells in the absence of NY-ESO-1 peptide pulsing at the point of T cell addition (126 h after seeding) and at the end of the assay (330 h) for peripheral blood PBL, CD4+ isolated, and CD8+ isolated T cell fractions. Black bars indicate mean 3D cell area. Two-tailed unpaired t-tests were performed comparing 3D cell area with NY-ESO-1^{c259} T vs. CD8α NY-ESO-1^{c259} T cells at 330h for all fractions and donors without peptide. The trend for improved killing reached statistical significance in the case of large spheroids at the 330 hr time point for wave 147 cells. As expected the CD8 fraction and PBL fraction (containing CD8+ cells) showed efficient killing of cells and no difference with CD8α co-expression.

1. A population of modified T cells that present an exogenous CD8 co-receptor or fragment thereof, and a T cell receptor (TCR).

2. A population of modified T cells of claim 1 wherein the CD8 co-receptor is CD8α.

3. A population of modified T cells of claim 2 wherein the CD8 co-receptor comprises an amino acid sequence having at least 80%, 85%, 90%, 95%, 96%, 97%, 98%, 99%, or 100% sequence identity to SEQ ID NO: 1.

4. A population of modified T cells of claim 1 wherein the TCR is an affinity enhanced TCR.

5. A population of modified T cells of claim 1 wherein the TCR is a NY-ESO-1 TCR.

6. A population of modified T cells of claim 5 wherein the TCR comprises an amino acid sequence having at least 80%, 85%, 90%, 95%, 96%, 97%, 98%, 99%, or 100% sequence identity to SEQ ID NO: 2.

7. A population of modified T cells of claim 5 wherein the TCR comprises an amino acid sequence having at least 80%, 85%, 90%, 95%, 96%, 97%, 98%, 99%, or 100% sequence identity to SEQ ID NO: 3.

8. A nucleic acid construct comprising;
i. a first nucleotide sequence encoding a CD8 co-receptor for fragment thereof; and
ii. a second nucleotide sequence encoding a T cell receptor.

9. A nucleic acid construct according to claim 8 wherein the CD8 co-receptor is CD8α.

10. A nucleic acid construct according to claim 9 wherein the nucleotide sequence encoding CD8α comprises a nucleic acid sequence having at least 80% sequence identity to SEQ ID NO: 4.

11. A nucleic acid construct of claim 8 wherein the TCR is an affinity enhanced TCR.

12. A nucleic acid construct according to claim 8 wherein the TCR is a NY-ESO-1 TCR.

13. A nucleic acid construct according to claim 12 wherein the TCR comprises a nucleic acid having at least 80%, 85%, 90%, 95%, 96%, 97%, 98%, 99%, or 100% sequence identity to SEQ ID NO: 5.

14. A nucleic acid construct according to claim 12 wherein the TCR comprises a nucleic acid having at least 80% sequence identity to SEQ ID NO: 6.

15. A vector comprising a nucleic acid construct according claim 8.

16. A vector according to claim 15 wherein the vector is a lentiviral vector.

17. A population of T cells comprising a nucleic acid construct according to claim 8.

18. A pharmaceutical composition comprising a population of T cells according to claim 1, and a pharmaceutically acceptable carrier.

19. (canceled)

20. A method for treating a subject afflicted with cancer, comprising administering to the subject a therapeutically effective amount of a pharmaceutical composition according to claim 18.

21. (canceled)

22. A method of engineering a modified T cell comprising:
i. Providing a T cell;
ii. Introducing the vector of claim 15 into said T cell; and
iii. Expressing said vector in the T cell.
